(19) United States
(12) Patent Application Publication
(10) Pub. No.: US 2025/0349405 A1
(43) Pub. Date: Nov. 13, 2025

(54) RADIOLOGY REPORT GENERATION SYSTEM AND METHOD

(71) Applicant: Lunit Inc., Seoul (KR)

(72) Inventors: Sang Hyup LEE, Seoul (KR); Taesoo KIM, Seoul (KR); Junha KIM, Seoul (KR); Gunhee NAM, Seoul (KR); Dillard LAURENT, Seoul (KR); Sanguk PARK, Seoul (KR); Weonsuk LEE, Seoul (KR); Hyeonsoo LEE, Seoul (KR); Junwoo CHO, Seoul (KR)

(21) Appl. No.: 19/000,942

(22) Filed: Dec. 24, 2024

(30) Foreign Application Priority Data
May 8, 2024 (KR) 10-2024-0060559

(51) Int. Cl. G16H 15/00; G06F 40/40; G06T 7/00; G06T 11/60

(52) U.S. Cl. CPC G16H 15/00; G06T 7/0014; G06T 11/60; G06F 40/40; G06T 2207/30096; G06T 2210/41

(57) ABSTRACT

A radiology report generation system is configured to obtain an analysis result for a target medical image using an artificial intelligence analysis model, extract at least one similar image to the target medical image from a catalog set comprising medical image-radiology report pairs; determine at least one radiology report paired with the at least one similar image as a reference image, and generate a radiology report for the target medical image based on the analysis result, using the reference report as a guideline.

### CROSS-REFERENCE TO RELATED APPLICATION

This application claims priority to and the benefit of Korean Patent Application No. 10-2024-0060559 filed in the Korean Intellectual Property Office on May 8, 2024, and Korean Patent Application No. 10-2024-0151239 filed in the Korean Intellectual Property Office on Oct. 30, 2024, the entire contents of which are incorporated herein by reference.

### BACKGROUND

### (a) Field

The present disclosure relates to generation of a radiology report.

### (b) Description of the Related Art

Recently, with the active introduction of Artificial Intelligence (AI) technology in the medical field, AI-based medical image analysis technologies, such as Lunit INSIGHT solutions, which analyze medical images and visually provides analysis results, are being studied.

Radiologists typically review images through a worklist, verify abnormalities identified by medical image analysis, and then create reports. There has been a growing interest in the development of technology that automatically generates radiology reports using generative artificial intelligence technology.

### SUMMARY

The present disclosure attempts to provide a system and method of generating a radiology report based on artificial intelligence.

The present disclosure also attempts to provide an interface screen that provides a radiology report.

An exemplary embodiment of the present disclosure provides a system for generating a radiology report, the system including: a memory; and a processor for executing instructions stored in the memory. The processor is configured to: obtain an analysis result for a target medical image using an artificial intelligence analysis model, extract at least one similar image to the target medical image from a catalog set comprising medical image-radiology report pairs; determine at least one radiology report paired with the at least one similar image as a reference report, and generate a radiology report for the target medical image based on the analysis result, using the reference report as a guideline.

The processor may be configured to: determine presence of findings corresponding to predetermined finding labels in the radiology report; and generate a finding label set with finding labels extracted from the radiology report.

The finding label set may be provided as a separate report distinct from the radiology report, or included in a designated section of the radiology report.

The processor may be configured to store a final radiology report, edited or confirmed for the radiology report by a user, in a designated location.

The processor may be configured to: determine whether to add the radiology report to the catalog set; and add a pair of the radiology report and the target medical image to the catalog set based on the determination.

The analysis result may include lesion information detected in the target medical image.

The analysis result may further include additional information extracted from the target medical image. The additional information may include at least one of detailed information on the detected lesion, information on additional findings other than the detected lesion, quality information on the target medical image, or information on metadata for the target medical image.

The processor may be configured to generate the additional information through visual question answering process, which extracts answers to questions in the target medical image.

The processor may be configured to: select a question set related to the target medical image or an analysis result of the target medical image from a question bank having questions; and extract an answer to each question included in the question set to generate the additional information.

The analysis result may further include quantitative information on an interest object present in the target medical image.

The processor may be configured to associate a non-text analysis result for the target medical image with the radiology report.

Another exemplary embodiment of the present disclosure provides a method of a radiology report generation by a system, the method including: obtaining an analysis result for a target medical image using an artificial intelligence analysis model; extracting at least one similar image to the target medical image from a catalog set comprising medical image-radiology report pairs; determining at least one radiology report paired with the at least one similar image as a reference report; and generating a radiology report for the target medical image based on the analysis result, using the reference report as a guideline.

The method may further include: determining presence of findings corresponding to predetermined finding labels in the radiology report; and generating a finding label set with finding labels extracted from the radiology report.

The method may further include: obtaining clinical information through user input or interworking with a database of a medical institution; and revising the radiology report using the clinical information or an analysis result of the clinical information.

The method may further include storing a final radiology report, edited or confirmed for the radiology report by a user, in a designated location.

The method may further include: determining whether to add the radiology report to the catalog set; and adding a pair of the radiology report and the target medical image to the catalog set based on the determination.

The analysis result may include at least one of lesion information detected in the target medical image, additional information extracted from the target image, or quantitative information on an interest object present in the target medical image. The additional information may include at least one of detailed information on the detected lesion, information on additional findings other than the detected lesion, quality information on the target medical image, or information on metadata for the target medical image.

The obtaining the analysis result may include: selecting a question set related to the target medical image or an analysis result of the target medical image from a question bank having questions, and extracting an answer to each question included in the question set to generate the additional information.

The method may further include associating a non-text analysis result for the target medical image with the radiology report.

Still another exemplary embodiment of the present disclosure provides a computer program stored in a computer-readable recording medium, the computer program comprising instructions to cause a processor configured to: obtain an analysis result for a target medical image using an artificial intelligence analysis model; extract at least one similar image to the target medical image from a catalog set comprising medical image-radiology report pairs; determine at least one radiology report paired with the at least one similar image as a reference report; and generate a radiology report for the target medical image based on the analysis result, using the reference report as a guideline.

According to the embodiment, a radiology report containing high reliability, accuracy and derailed analysis information can be automatically generated by using lesion information detected in a medical image, including detailed information, such as lesion location information, lesion severity, additional findings beyond the detected lesions, information on medical image quality and metadata, and quantitative information on interest objects such as lesions.

According to the exemplary embodiment, by generating a radiology report using a reference report as a guideline, it is possible to generate a radiology report that ensures reliability and accuracy, while also reducing errors caused by Hallucination, a common issue with the generative artificial intelligence model.

According to the exemplary embodiment, by managing reference reports as a catalog set for use when generating a radiology report, it is possible to generate a radiology report customized to the user's writing style or preference.

According to the exemplary embodiment, by formalizing the radiology report into a finding label set, the clinical validity of the radiology report can be evaluated using the finding label set, thereby enhancing the reliability of the radiology report generated through artificial intelligence.

According to the exemplary embodiment, by associating a secondary image that visually provides lesion information with the text radiology report, the user's understanding of the radiology report may be enhanced, thereby improving reading efficiency.

According to the exemplary embodiment, by automatically generating a radiology report based on the analysis result for a medical image, reading efficiency may be increased by reducing the user's reading time and workload, and as a result, memory and computing resources of a medical imaging system used for managing medical images awaiting review may be optimized.

### BRIEF DESCRIPTION OF THE DRAWINGS

FIG. 1 is a diagram illustrating a concept of a radiology report generation system according to an exemplary embodiment.

FIG. 2 is a diagram of a radiology report generation system according to the exemplary embodiment.

FIG. 3 is a diagram illustrating an example of a radiology report according to the exemplary embodiment.

FIG. 4 is a diagram illustrating a method of generating a finding label set according to the exemplary embodiment.

FIG. 5 is a diagram illustrating a radiology report generation method according to an exemplary embodiment.

FIG. 6 is a diagram illustrating a visual question and answer (VQA) process of an additional information extractor according to the exemplary embodiment.

FIG. 7 is a diagram illustrating an example of a radiology report providing method according to an exemplary embodiment.

FIG. 8 is a flow diagram of the radiology report generation method according to the exemplary embodiment.

FIG. 9 is a flowchart of a method of revising a radiology report according to the exemplary embodiment.

FIG. 10 is a flowchart of a method of managing a catalog set according to the exemplary embodiment.

### DETAILED DESCRIPTION OF THE EXEMPLARY EMBODIMENTS

Hereinafter, the present disclosure will be described more fully hereinafter with reference to the accompanying drawings, in which exemplary embodiments of the invention are illustrated. As those skilled in the art would realize, the described exemplary embodiments may be modified in various different ways, all without departing from the spirit or scope of the present disclosure. Accordingly, the drawings and description are to be regarded as illustrative in nature and not restrictive. Like reference numerals designate like elements throughout the specification.

In addition, unless explicitly described to the contrary, the word “comprise”, and variations such as “comprises” or “comprising”, will be understood to imply the inclusion of stated elements but not the exclusion of any other elements. In addition, the terms “-er”, “-or”, and “module” described in the specification mean units for processing at least one function and operation, and may be implemented by hardware components or software components, and combinations thereof.

A device or terminal of the present disclosure is a computing device configured and connected to at least one processor to perform the operations of the present disclosure by executing instructions. A computer program may include instructions written to cause the processor to execute the operations of the present disclosure and may be stored on a non-transitory computer readable storage medium. The computer program may be downloaded over a network or sold as a product.

A medical image of the present disclosure may be an image of a patient's body part taken by using various modalities or may be a pathology image. For example, the modalities may include x-ray, magnetic resonance imaging (MRI), ultrasound, computed tomography (CT), mammography (MMG), digital breast tomosynthesis (DBT), endoscopy, positron emission tomography (PET), and the like, and the medical images obtained thereby may include X-ray images, MRI images, ultrasound images, CT images, MMG images, DBT images, endoscopy images, PET images, and the like.

A user of the present disclosure may be a healthcare professional, such as, but not limited to, a doctor, nurse, clinical pathologist, radiologist, sonographer, or medical imaging specialist (radiologist).

An artificial intelligence model (AI model) of the present disclosure is a model for learning at least one task, which may be executed by a processor. The task that the AI model learns may refer to a problem to be solved through learning or a task to be performed through learning. The artificial intelligence model may be implemented as a computer program executed on a computing device, may be downloaded over a network, or may be sold in a product form. Alternatively, the artificial intelligence model may interwork with various devices through a network.

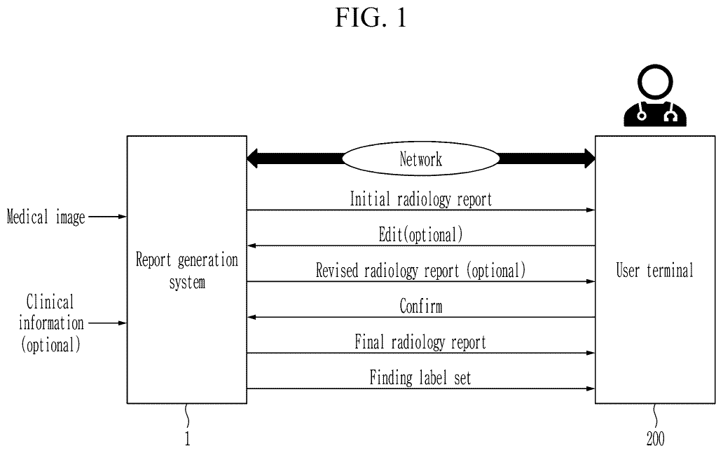

FIG. 1 is a diagram illustrating a concept of a radiology report generation system according to an exemplary embodiment.

Referring to FIG. 1, a radiology report generation system (referred to simply as an “system”) 1 is a computing device that is implemented to analyze a medical image using an artificial intelligence model and automatically generate a radiology report based on the analysis results. The radiology report generated by the system 1, the information derived from the radiology report (e.g., finding label set), or the analysis results (e.g., lesion information) used to generate the radiology report, may be provided to a user terminal 200 through a network. The medical image may be obtained by imaging a body part of a patient using various modalities or may be a pathological image. For example, the medical image may be an X-ray image, an MRI image, an ultrasound image, a CT image, an endoscopic image, and a PET image, classified according to the imaging device, and may include a chest X-ray image, an MMG image, a DBT image, and the like, classified according to the body part imaged. In the description, the radiology report may be simply referred to as a report.

The system 1 may generate a radiology report by additionally using clinical information of a patient provided with a medical image. The clinical information may be used for various purposes during a radiology report generation procedure. The system 1 may obtain various clinical information of a patient by interworking with various databases of a medical institution, such as a picture archiving and communication system (PACS), an electronic medical record (EMR), an electronic health record (EHR). In addition, information generated by the system 1 may be stored in a designated database within a medical institution.

The user terminal 200 may provide a user interface that displays relevant information on a screen in conjunction with the system 1 or a database storing medical data. The user terminal 200 may display information generated by analyzing a medical image in the system 1 through a dedicated viewer.

The system 1 may be a server device, and the user terminal 200 may be a client terminal installed in a medical institution, and the system 1 and the user terminal may interwork through a network. The system 1 may be a local server connected to a network within a specific medical institution. Alternatively, the system 1 may be a cloud server and may interwork with terminals (medical staff terminals) of a plurality of medical institutions having access rights. The system 1 may be a cloud server and may interwork with a patient's individual terminal having access rights.

The system 1 may generate the radiology report using the analysis result processed by an artificial intelligence model. The system 1 may generate the radiology report from the analysis result for the medical image by employing a language model. The analysis result for the medical image may include lesion information detected from the medical image, additional findings other than a specific lesion, quality and metadata of the medical image (e.g., modality, and imaging details), quantitative information extracted from the medical image (e.g., size, volume, ratio, and number of lesions) and the like.

The system 1 may generate a radiology report by referencing a report of a similar image during the report generation process. By using the report of the similar image as the guideline, the system 1 may enhance the reliability and accuracy of the generated radiology report, and reduce errors caused by hallucination, a common issue with the generative artificial intelligence model.

The system 1 may generate a radiology report including both non-text and text-based analysis results by associating the non-text analysis result with the text-based analysis result for the medical image. The non-text analysis result may include, for example, a secondary image that visually provides the analysis result including lesion information. The secondary image may be generated, for example, as DICOM secondary capture (SC). Accordingly, the system 1 may enhances the explainability of the report by incorporating both AI-driven text and visual elements.

The system 1 may generate a final radiology report through user confirmation on an initial radiology report. The initial radiology report may be edited by a user, and a final radiology report may be generated after the user confirms the revised report. An editing process by the user may be performed selectively. Accordingly, the initial radiology report may be stored as the final radiology report upon user confirmation. Alternatively, the system 1 may generate the initial radiology report, and then revise the initial radiology report using additional clinical information to regenerate the radiology report. The user confirmation procedure may be omitted, and the initial radiology report may be stored as the final radiology report.

The system 1 may generate a finding label set from the radiology report, composed of predefined finding labels. The finding label set indicates whether a predefined clinical finding is present in the analysis result for a medical image. The finding label set may be used to evaluate the clinical validity of the radiology report. A radiology report generated using a large language model (LLM) expresses the same finding in various phrases, which can lead to inconsistencies in the report and make it difficult to quantitatively evaluate the clinical performance of the system 1 from the radiology report that is the result of the system 1. To address this, the content of the radiology report is formalized into predefined finding labels to generate a finding label set, which may be used to evaluate performance, such as reliability, reproducibility, sensitivity, and specificity, of the radiology report generation. Further, through the finding label set allows users to quickly identify the main findings detected in the medical image.

The radiology report and the finding label set generated by the system 1 may be transmitted to the user terminal 200 or a designated device. The finding label set may be provided as a separate radiology report distinct from the radiology report, or may be included in a designated section of the radiology report. Alternatively, the finding label set may be provided as part of the analysis result of the medical image, such as in DICOM SC. In the description, the radiology report and the finding label set are described separately, and this description does not exclude the possibility of the finding label set being included in the radiology report. The method of providing the finding label set may vary depending on the specific settings.

The clinical information may be used for various purposes during a radiology report generation process. The system 1 may either generate an analysis result used to generate a radiology report using clinical information or may revise the radiology report using the clinical information. Clinical information may be obtained through a user input or interworking with a database storing clinical information. The system 1 may provide an interface for users to input clinical information. For example, a user may input clinical information through an input device, such as a keyboard, a mouse, or a microphone. The system 1 may actively obtain clinical information necessary for generating a radiology report. That is, the system 1 may inquire about the presence or absence of clinical information necessary for generating a radiology report based on the analysis result for the medical image and receive clinical information from the user. For example, in response to the detection of specific findings in a medical image, the system 1 may prompt the user to provide relevant clinical information to the specific findings and generate a radiology report using the input clinical information. Alternatively, the system 1 may search for clinical information related to the specific findings in the database of a medical institution in response to the detection of specific findings in a medical image.

The clinical information used to generate the radiology report may be diverse and may include multimodal data such as text and images. The clinical information may include, for example, text data detailing a patient's symptoms, test results (e.g., blood test, function test results), age, gender, and reason for examination of the patient. The clinical information may include, for example, an additional image other than the target image for which the radiology report is being generated. The additional image may include a past image taken with the same type of imaging device as the target image, or an image taken with a different type of imaging device from the target image.

For convenience of description, although the present disclosure is described in a simple form in which the user terminal 200 and the system 1 communicate with each other, the procedure for the user terminal 200 to receive the radiology report generated in the system 1 and to revise and confirm the received radiology report may be implemented through interworking of various devices. For example, the user terminal 200 may display the radiology report and receive the user input through a viewer displaying medical image-related data. The viewer may be installed and executed, for example, in a computing device within a workstation, and may include a PACS viewer interworking with a picture archiving and communication system (PACS), but is not limited thereto.

In the following, the operation of the radiology report generation system is described in detail.

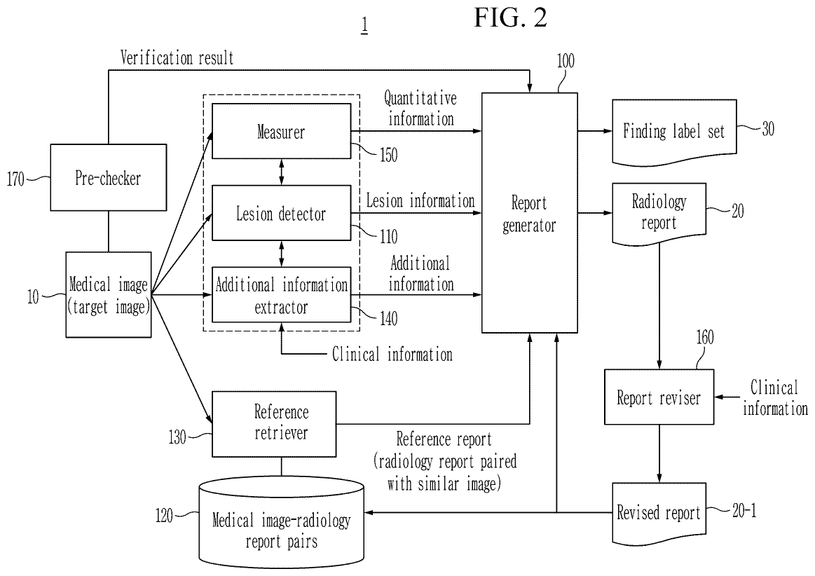

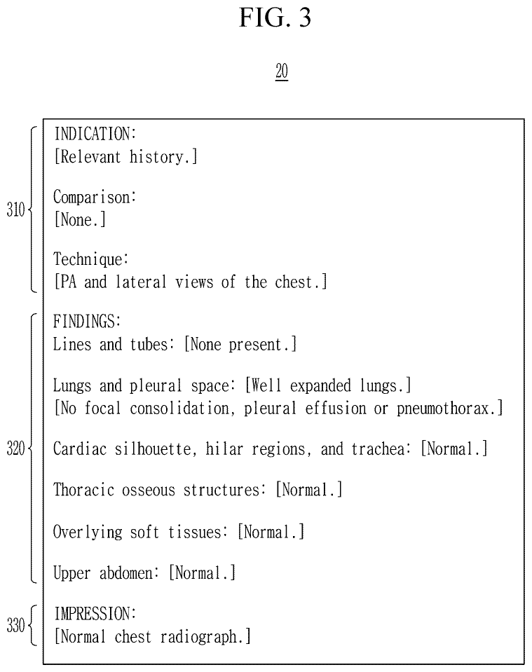

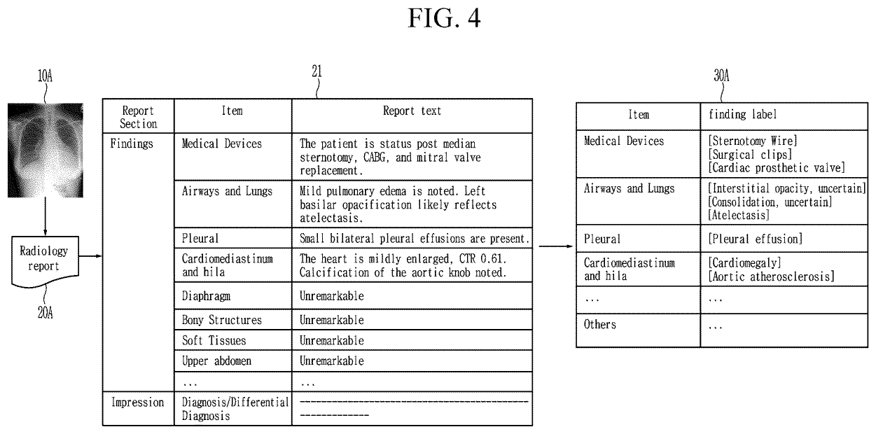

FIG. 2 is a diagram of the radiology report generation system according to the exemplary embodiment, FIG. 3 is a diagram illustrating an example of a radiology report according to the exemplary embodiment, and FIG. 4 is a diagram illustrating a method of generating a finding label set according to the exemplary embodiment.

Referring to FIG. 2, the system 1 may be executed by at least one processor, and include a report generator 100 that generates a radiology report 20 based on an analysis result for a medical image 10, a lesion detector 110 that generates an analysis result for the medical image 10, a reference storage 120 that stores medical image-radiology report pairs which serve as a reference or a guideline, and a reference retrieval module 130 that extracts a radiology report mapped to a similar image of the medical image 10 from the reference storage 120. Here, the report generator 100 may generate a finding label set 30 with predefined finding labels, from the radiology report 20. The system 1 may provide the radiology report and the finding label set to the outside, and may include an interface device (not illustrated) for obtaining an input related to the radiology report.

Meanwhile, the system 1 may further include an additional information extractor 140 that provides additional analysis results for the medical image 10, and a measurer 150 that extracts quantitative information (e.g., size, volume, ratio, and number of lesions) of an interest object from the medical image 10.

The lesion detector 110, the additional information extractor 140, and the measurer 150 may be implemented as an artificial intelligence analysis model that outputs analysis results for the medical image 10 or clinical information, and may be designed to use analysis results generated for other models. At least one analysis result obtained by the lesion detector 110, the additional information extractor 140, and the measurer 150 may be used to generate a radiology report. In order to distinguish and explain the method of analyzing the medical image 10 or information extracted from the medical image 10, the artificial intelligence analysis model is divided into the lesion detector 110, an additional information extractor 140, and the measurer 150, and does not need to be clearly distinguished physically or logically, and may be implemented as at least one artificial intelligence model. In addition, although the present disclosure is described based on the case where the system 1 includes an artificial intelligence analysis model that analyzes medical images, the lesion detector 110, the additional information extractor 140, or the measurer 150 need not be a dedicated model for the system 1.

The system 1 may further include a report reviser 160 for generating a revised report 20-1 by revising the radiology report 20 generated by the report generator 100.

In addition, the system 1 may further include a pre-checker 170 that verifies the suitability of the medical image 10 before inputting the medical image 10 to the lesion detector 110 or the like. The pre-checker filters out medical images that are inappropriate for analysis by the artificial intelligence model, ensuring the reliability of the analysis result, and consequently, enhancing the reliability of the radiology report.

In addition to the lesion detector 110, the additional information extractor 140, and the measurer 150, the components constituting the system 1 are blocks named to distinguish and explain the operation, and need not be clearly distinguished physically or logically, and may be implemented as at least one artificial intelligence model.

The report generator 100 is implemented to receive the analysis result for the medical image 10 and a radiology report (reference report) paired with a medical image similar to the medical image 10, and to generate a radiology report based on the analysis result using the reference report as a guideline. The report generator 100 may be implemented as a large language model that generates a radiology report by receiving the analysis result and the reference report as a prompt, but is not necessarily limited thereto. The report generator 100 is implemented to interwork with an external language model, and may transmit a command through an interface with an external language model, and obtain a radiology report generated through the same.

The analysis result of the medical image 10 may include predetermined lesion information, additional information extracted from the medical image 10, quantitative information (e.g., size, volume, ratio, or number) on an interest object (e.g., lesion, organ, and medical device), and may be obtained by using at least one of the lesion detector 110, the additional information extractor 140, and the measurer 150. Here, the additional information may include detailed information on the detected lesion (e.g., location information, detailed information (e.g., severity), information on various additional findings other than the detected lesion, information on the quality of the medical image, and metadata, and the like, and may be obtained through a visual question answering (VQA) process that extracts answers to questions from a given medical image.

The lesion detector 110 may be an AI model trained to perform a task of detecting an abnormal finding (e.g., lesion) in a medical image. The lesion detector 110 may be a medical image analysis model including a computer-aided diagnostic (CAD) model. The likelihood of existence of predefined abnormal findings may be generated as a continuous value in the range of 0 to 1, and may be converted into a binary value indicating the presence of abnormal findings when it exceeds or equals a threshold. The lesion detector 110 may output, as lesion information, a secondary image indicating a list of detected lesions from the medical image 10 and the detected lesion information as a heat map.

The analysis result of the medical image provided by the lesion detector 110 may be provided in various formats, for example, in a DICOM format, a secondary capture (SC), a gray scale soft copy presentation state (GSPS), or a structured radiology report (SR). The SC may be generated separately from the original medical image and display the analysis result (e.g., a lesion score indicating the presence or absence of the lesion, and a lesion location) as a heatmap, a contour, and the like. The GSPS is configured to overlay and display lesion information on the original medical image, and may turn on/off the overlaid lesion information. For example, the lesion detector 110 may detect lesion information from a chest X-ray image by analyzing the chest X-ray image. The lesion detector 110 may detect predetermined lesions from the chest X-ray image, such as nodule, pneumothorax, pleural effusion, consolidation, cardiomegaly, atelectasis, pneumoperitoneum, calcification, fibrosis, mediastinal widening, tuberculosis, and acute bone fracture. The lesion detector 110 may calculate other major medical indexes, such as abnormal score.

The lesion information, which is an analysis result generated by the lesion detector 110, is provided to the report generator 100, and may be provided to other artificial intelligence analysis models to help other artificial intelligence analysis models to analyze. For example, a lesion list may be provided to the additional information extractor 140, and the additional information extractor 140 is used to identify a detected lesion in a medical image, select questions for obtaining additional information on the detected lesion, select questions for detecting additional findings other than the detected lesion, or obtain clinical information related to the detected lesion. Likewise, a lesion list may be provided to the measurer 150, and the measurer 150 may be used to identify a lesion detected in a medical image and measure quantitative information on the lesion. In addition, a secondary image (e.g., SC image) output from the lesion detector 110 may be provided to the additional information extractor 140 or the measurer 150.

The additional information extractor 140 is a model that complements the lesion detector 110 that detects the specified lesion, and may be an AI model trained to extract additional information (e.g., location) about the lesion detected by the lesion detector 110, or to extract various additional information other than the detected lesion. The additional information extractor 140 may include a visual question answering-based language model. The training method of the additional information extractor 140 may be various, for example, trained through contrastive learning using multi-modal data including medical images and clinical information of a patient.

The additional information extractor 140 may be implemented based on a foundation model. A foundation model is an unsupervised or self-supervised model trained with large-scale data that requires little or no annotated data, and generally learns useful information by using unlabeled large-scale data. For example, the foundation model may be train using paired image and text descriptions (e.g., medical image and radiology report) on a patient basis. Instead of regressing answers (annotations) from a given image, a foundation model may be trained by comparing data. For example, when paired image and text descriptions are given, the training objective is to find the representation (embedding space) that best facilitates comparing data. In particular, contrastive learning may be used in which the features of the correct image-radiology report pair are made more similar to each other than the features of the random pair.

The additional information extractor 140 may receive the medical image 10 and extract detailed information related to the medical image. In this case, the additional information extractor 140 may receive clinical information together with the medical image and extract additional information from the multi-modal data. In addition, the additional information extractor 140 may receive an analysis result provided by the lesion detector 110 or the measurer 150 and analyze additional information on the medical image 10 by using the analysis result. The additional information extractor 140 may extract detailed information related to the medical image through a visual question answering (VQA) process and to this end, the additional information extractor 140 may be implemented to obtain answers to questions from the medical image 10 or clinical information. The additional information extractor 140 may output additional information including a question and answer pair for the medical image 10 as an analysis result. The analysis result provided by the additional information extractor 140 may be variously determined according to questions.

The additional information extractor 140 may select questions used to obtain additional information according to the attributes of the medical image, body parts, patient information, lesion information or quantitative information detected in the medical image. The additional information extractor 140 may select a medical image and a question set related to the analysis result thereof from a question bank composed of questions, or may generate a question set necessary for generating a radiology report. The analysis result of the medical image may be obtained through interworking with the lesion detector 110 or the measurer 150, or may be obtained through self-analysis.

Additional information provided by the additional information extractor 140 may be variously determined according to the questions. For example, the additional information extractor 140 may perform analysis on the medical image 10 or clinical information to answer the questions related to the location or lesion state (e.g., severity or size) of the detected lesion, medical device (e.g., catheter or artificial pacemaker) or surgical site, clinical findings, and the like. In addition, for questions regarding the quality or the metadata of the medical image 10, the additional information extractor 140 may perform analysis, such as quality assessment or metadata check on the medical image 10 to provide corresponding answers. For example, the additional information extractor 140 may analyze the medical image 10 or clinical information to answer specific questions, such as Table 1 and output additional information in the form of answers to the questions as an analysis result. In Table 1, ‘FINDING_NAME’ represents various findings detectable in a medical image. This may include lesions or medical devices that were not detected by the lesion detector 110.

TABLE 1
Examples of Question

Is the image acquired with adequate quality for interpretation?
Is this a frontal view chest x-ray?
Is this a frontal view chest x-ray from a pediatric case?
Is there a {FINDING_NAME} in the upper-left lung zone?
Is there a {FINDING_NAME} in the upper-right lung zone?
Is there a {FINDING_NAME} in the lower-right lung zone?
Is there a {FINDING_NAME} in the lower-left lung zone?
Is the {FINDING_NAME} severe?

A question set for additional analysis of the medical image 10 may be configured in various ways. For example, the question bank may include various questions, such as: “Is there a nodule in the upper-right lung zone?”, “Is there a rib-fracture in the left fourth rib?”, “′ Is the image quality adequate?” and “Is this a front view chest X-ray?”. Thereafter, the additional information extractor 140 may select a set for questions from the question bank to obtain detailed additional information from the medical image 10, based on the lesion list detected in the medical image 10. For example, when a nodule is detected in the medical image 10 and a rib-fracture is not detected, the additional information extractor 140 may select a detected lesion-related question such as “Is there a nodule in the upper-right lung zone?” while excluding non-detected lesion-related questions such as “Is there a rib-fracture in the left fourth rib?”.

When a question set for a medical image is determined, the additional information extractor 140 may analyze the given information and output a text-based answer for each question. The additional information extractor 140 may obtain answers to the questions using a vision language model trained through visual instruction tuning.

The measurer 150 may be an AI model trained to analyze quantitative information (e.g., size, volume, ratio, and number) for each measurement item on an interest object (e.g., lesion, organ, and medical device) in the medical image 10. In this case, the measurer 150 may directly measure quantitative information of the interest object from the original medical image 10, or may measure quantitative information of the interest object from an auxiliary image (e.g., SC image) including lesion information. The items to be measured by the measurer 150 may be determined according to an interest object, attributes of a medical image, a body part, or the like, or may be determined according to the lesion information or the additional information detected from the medical image. The lesion information or additional information detected from the medical image may be obtained through interworking with the lesion detector 110 or the additional information extractor 140, or may be obtained through self-analysis. The quantitative information on the interest object may be a measured value to be included in a radiology report, such as a lesion, an organ, and a medical device. The quantitative information on the interest object may include, for example, a lesion size, organ volume, a cardiothoracic ratio, an endotracheal (ET) tube to carina distance, and the like.

The measurer 150 may receive an analysis result from the lesion detector 110 or the additional information extractor 140, identify an interest object (e.g., nodule) requiring quantitative measurement from the analysis result, and measure a measurement item (e.g., size) designated for the interest object. The measurer 150 may provide metric measurements for the interest objects. For example, the measurer 150 may generate quantitative information with an interest object, a measurement value, and a measurement unit in a format such as <nodule: 3.0, mm>.

Meanwhile, the lesion detector 110, the additional information extractor 140, or the measurer 150 implemented to analyze the medical image may variously provide an analysis result of the medical image, and may output an analysis result visually displayed together with an analysis result in a text form. For example, the lesion detector 110 may provide an analysis result of the medical image to DICOM SC, GSPS, SR, or the like.

The text-based analysis results provided by the lesion detector 110, the additional information extractor 140, or the measurer 150 are input to the report generator 100 and used to generate the radiology report. The non-text analysis result may be provided with the radiology report. The system 1 may be implemented to provide an analysis result with an auxiliary image (e.g., SC image) that visually displays text description of the lesion mentioned in the radiology report and the corresponding lesion. The method of providing the radiology report and the visually displayed analysis result may be implemented in various ways, and for example, when the user clicks on or hovers over the lesion name in the radiology report displayed through the viewer of the user terminal 200, the SC image including the lesion information may be displayed with the radiology report.

The reference storage 120 stores medical image-radiology report pairs to serve as guidelines for generating radiology reports. Each medical image-radiology report pair may include annotations related to the presence and location of findings. The reference storage 120 may manage a catalog set consisting of N number of predefined medical image-radiology report pairs. In this case, each medical image may be processed through an encoder to generated into a feature representation vector through an encoder and stored. The medical images and radiology reports stored in the reference storage 120 may be referred to as reference images and reference reports.

The random radiology report stored in the reference storage 120 serves as guidelines for generating new radiology report. Accordingly, the user may add or change the catalog set to customize the radiology report generated by referencing these stored reports. For example, the user may add a radiology report written in a format adopted by their medical institution to the catalog set, add a radiology report focused on lesions or findings commonly detected at their medical institutions to the catalog set, add a radiology report addressing rare cases to the catalog set, or add a radiology report styled in a specific manner preferred by the user to the catalog set. In addition, the user may add a radiology report generated and confirmed by the report generator 100 to the catalog set. This allows users to enrich the reference pool, ensuring that the report generator 100 can access tailored references for generating new radiology reports.

According to another exemplary embodiment, the catalog set may be managed without user intervention or with minimal user intervention. For example, a device designated to manage the reference storage 120, for example, the reference retrieval module 130 may determine whether it is useful to add the final radiology report to the catalog set. This determination the usefulness of the new radiology report can be based on the potential information gain from adding the new report to the catalog set. The reference retriever 130 may estimate the information gain using a trained machine learning algorithm, such as a neural network, that outputs a score for the information gain. According to another exemplary embodiment, the reference retriever 130 may embed the radiology reports as feature representation vectors and evaluate the similarity between the reference reports stored in the catalog set and the new radiology report. A new radiology report with low similarity to the stored the references in the catalog set, as determined by similarity distance between the new radiology report and the reference report, may be added to the catalog set. According to another exemplary embodiment, the reference retriever 130 may determine whether to add the radiology report generated by report generator 100 to the catalog set based on the characteristics of the catalog set such as the number of cases included in the catalog set and the feature distribution of the cases (e.g., lesion, and other findings distribution) included in the catalog set.

There may be various methods of adding a new radiology report to the catalog set. The reference retriever 130 adds the radiology report generated by the report generator 100 to the catalog set until M cases are filled in the catalog set. After filling the catalog set with M cases, the reference retriever 130 may determine whether the radiology report newly generated by the report generator 100 differs from the cases included in the catalog set, discard the case similar to the other case among the M cases included in the catalog set according to the determination result, and add a new case. That is, since duplicate medical image-radiology report pairs are unnecessary in the catalog set, the reference retriever 130 may prioritize adding rare cases such as those involving uncommon lesions or findings if they are found based on the feature distribution of the cases included in the catalog set.

The reference retriever 130 may identify at least one image similar to the medical image 10 among the reference images stored in the reference storage 120 and select the radiology report paired with the similar image as a reference report. The reference retriever 130 may provide the reference report to the report generator 100, or may provide a pair of the similar image and the reference report to the report generator 100. Information provided to the report generator 100 may be referred to as guideline information, and guideline setting, such as the number of reference reports and whether a similar image is provided may be determined according to the report generator 100. Specifically, when the target image 10 for generating the radiology report is provided, the reference retriever 130 may generate a feature representation vector of the target image 10 through an encoder. The reference retriever 130 may determine K reference image-report pairs similar to the target image by comparing similarities between the vector of the target image 10 and the vectors of the reference images stored in the reference storage 120. According to the guideline settings, K reference image-report pairs may be output as guideline information, or K radiology reports may be output as guideline information. The similarity may be calculated in various ways including cosine similarity.

Since the K guidelines are most similar data to the target image, the K guidelines may serve as a reference in the process of generating a radiology report. That is, the report generator 100 may generate a radiology report with comprehensive conclusions, similar to one prepared by a radiologist by using the K reference reports as a guideline. This helps reduce errors caused by hallucinations, a common issue with the generative artificial intelligence model.

The report generator 100 may obtain an analysis result for the medical image 10. The analysis result for the medical image 10 may be provided from the lesion detector 110, the additional information extractor 140, and the measurer 150. The analysis result on the medical image 10 may include lesion information detected from the medical image 10, additional information with answers obtained by analyzing the medical image 10 or clinical information on the question, and quantitative information on an interest object included in the medical image 10. The report generator 100 may obtain a reference report written in advance for an image similar to the medical image 10. The reference report may be provided from the reference retriever 130.

The report generator 100 may generate a radiology report from the analysis result for the medical image 10. The report generator 100 may be an AI model (e.g., large language model (LLM)) implemented to generate a radiology report in a standardized description method and structure for each section, similar to the radiology reports prepared by medical experts. The report generator 100 may generate a radiology report for the medical image 10 in a structured format based on the analysis result for the medical image 10 with at least one reference report.

The format of the radiology report generated by the report generator 100 may vary, and with configurable options that allow for adjustments to the amount of text, a description style, a written content, and the like included in the radiology report. In addition, the configuration may enable the selection of different formats, such as a full version radiology report with detailed analysis or a short version radiology report focusing on concise analysis for essential items. The report generator 100 may generate a radiology report in a designated format. Meanwhile, the report generator 100 may generate radiology reports in a plurality of formats based on the analysis of the medical image 10. Initially, the radiology report in a selected format may be provided to the user, and if the user selects for a different format, the report may be quickly reformatted and provided in the new format.

The report generator 100 may provide an interface that allows a user to input settings for generating a radiology report. The user may create a user-defined format in the interface provided through the user terminal 200, and may personalize the radiology report generation method such as text amount, description method, description content, and the like. In addition, the user may select a desired format from various available radiology report formats in the interface provided through the user terminal 200. To this end, the report generator 100 may provide a variety of radiology report formats based on the features of each medical image (e.g., modality and body parts), and may generate a radiology report in a format selected by a user from among the provided radiology report formats.

Referring to FIG. 3, the radiology report 20 may be structured into, for example, a header section 310, a finding section 320, and a clinical estimation section 330. The header section 310 may include, for example, items of INDICATION, COMPARISON, and TECHNIQUE. The header section 310 may be generated to include quality and metadata (e.g., imaging information such as modality, view position, and the like) of the medical image 10, and comparison information with a past image. The finding section 320 may be generated to include specific lesion information analyzed by the medical image 10 and a detailed description of various findings. The clinical estimation section 330 may include the description of a comprehensive conclusion, summary, differential diagnosis DDx, and recommended action on the medical image 10.

The report generator 100 may write text in the header section 310 by using the quality and metadata (e.g., modality information, imaging information such as view position, and the like) of the medical image 10 included in the analysis result. The quality of the medical image 10 may be included in an answer obtained by the additional information extractor 140 through analyzing the medical image 10, in response to a question such as “Is the image acquired with adequate quality for interpretation?” to inquir the quality of the given medical image. The metadata of the medical image 10 may be, for example, included in an answer obtained by the additional information extractor 140 through analyzing metadata of the medical image, in response to a question such as “Is this a frontal view chest x-ray?”.

The report generator 100 may describe in the finding section 320 a description generated by using specific lesion information included in the analysis result, various findings, and quantitative information on an object such as lesion, organs, and medical devices. The finding section 320 may be generated to include findings detailed for each anatomical structure and other items present in the image.

The report generator 100 may infer a comprehensive conclusion, summary, differential diagnosis (DDx), recommended action, and the like based on the analysis result of the medical image 10, and write the inference result in the clinical estimation section 330.

The report generator 100 may generate instructions (commands) for generating a radiology report using the analysis result of the medical image 10 and the reference report, and may obtain a radiology report by applying the instruction to the language model (e.g. LLM). This prompt process may be implemented to apply predetermined questions to the language model in order, or to apply questions adaptively generated according to the analysis result to the language model.

For example, the instruction for generating a radiology report may include instruction such as “The input image contains the following findings: [finding 1 located at X1 with severity of Y1, finding 2 located at X2, with measurement of 3 mm . . . ]. A similar x-ray sample has the following radiology report: {reference radiology report text goes here}. Please generate a radiology report.”, to generate a radiology report for the medical image 10 using the analysis result and the reference report.

For another example, the instruction statement for generating the radiology report may include a system prompt and a user prompt as illustrated in Table 2. The report generator 100 may write a system prompt that implies that the language model is an excellent medical imaging expert radiologist and gives the language model a role in generating a radiology report by collecting a lot of given information. The report generator 100 may write a user prompt by generating, based on the analysis result of the medical image 10, sentences indicative of lesion information detected in the medical image 10, additional information analyzed in the medical image 10 (e.g., quality, metadata, and various findings), quantitative information on an object included in the medical image 10, and the like, statements indicative of K-reference reports, and sentences instructing to generate a radiology report in a specific format by collecting given information.

TABLE 2

| System | You are an expert radiologist and will generate reports | |
|---|---|---|
| prompt | following ACR guidelines. Your replies should only contain | |
| | the requested information and nothing else. | |
| User | Sentences indicative | This is a vector of abnormal scores from |
| prompt | of lesion | an AI CAD engine: {FINDING_1: |
| | information | SCORE_1, FINDING_2: SCORE_2, |
| | | . . . , FINDING_N: SCORE_N}. |
| | Sentences indicative | The imaging acquisition quality is |
| | of additional | adequate. |
| | information (quality, | This is a frontal view chest X-ray. |
| | view position, and | The upper-right lung zone is normal, . . . |
| | findings) | There is no medical device to provide |
| | | measurements. |
| | Sentences indicative | This is the first similar reference radiology |
| | of reference report | report: {REFERENCE REPORT 1 from |
| | | Retrieval Module}. This is the second |
| | | similar reference radiology report: |
| | | {REFERENCE REPORT 2 from Retrieval |
| | | Module}. |
| | Sentences to instruct | Please write the radiology report given the |
| | to generate report | information. |

The language model receives instruction statements composed of sentences as in Table 2 and generates a radiology report, and in particular, generate a radiology report of the medical image 10 by referencing a radiology report of an image similar to the medical image 10. In this case, the language model may display and output reference information including at least one of content, features, information, and sources of the reference report on the radiology report. In addition, when the language model generates a radiology report by referring to patient history information (e.g., EMR/EHR data, past radiology report), reference information including patient history information may be displayed and output on the radiology report.

The report generator 100 may determine the presence or absence of findings corresponding to predetermined finding labels in the final radiology report, and generate a finding label set 30 with finding labels extracted from the final radiology report. The finding label set 30 may consist only of finding labels extracted from the radiology report, or may distinguish and display labels that exists and labels that does not exist in the radiology report among all predefined finding labels.

The finding label set 30 may indicate whether predefined clinical findings exist in the analysis result or a medical image and may be used to evaluate the clinical effectiveness of the radiology report. The finding label set 30 may be provided as a separate radiology report distinguished from the radiology report 20, or may be included in a designated section (e.g., finding label section) of the radiology report 20.

The report generator 100 may extract a finding label corresponding to the content of the radiology report by using a language model, and through this, generate a finding label set 30 with finding labels extracted from among all predefined finding labels. For example, the report generator 100 may simply extract labels corresponding to findings explicitly present in the sentences of the radiology report, or may extract finding labels through the semantic analysis of text included in the radiology report. When the presence of a specific finding in the text included in the radiology report is uncertain, the report generator 100 may add information indicating that the presence of the specific finding is uncertain to the corresponding label.

The finding label included in the finding label set 30 indicates that the corresponding findings are present in the radiology report, and the finding label may further include additional information, such as the location or uncertainty of the corresponding findings. Meanwhile, finding labels extractable from the radiology report may be defined for each findings item. The findings item may be classified according to a certain criterion (e.g., anatomical section, medical device). The finding label set 30 may be structured into findings items to which the finding labels belong. That is, the finding label set 30 may be structured to recognize a finding label extracted from the radiology report among all labels defined in each findings item.

The report generator 100 may predefine the finding items and finding labels to be extracted from the radiology report for each image type, and generate a consistent finding label set 30 by using the finding items and finding labels designated for the type of medical image.

The generation of a finding label set when the medical image is a chest X-ray image 10A will be described with reference to FIG. 4. The finding label set for the chest X-ray image may be generated based on, for example, a medical device, airways and lungs, pleural, Cardiomediastinum and hila, a diaphragm, bony structures, soft tissues, upper abdomen, and the like, and finding labels extractable from each findings item.

The report generator 100 may generate a radiology report 20A by using the analysis result of the chest X-ray image 10A, and may generate a finding label set 30A by extracting finding labels corresponding to the text of the radiology report 20A. The text of the radiology report 20A may be mapped for each findings item, and a findings item mapping table 21 is presented to explain a process of generating the finding label set 30A from the radiology report 20A, and the finding label set may not necessarily be generated only when the findings item mapping table 21 is generated.

The report generator 100 may extract corresponding finding labels, such as [Sternotomy Wire], [Surgery Clip], [Cardiac prosthetic valve] from texts about medical devices such as “The patient is status post median sternotomy, CABG, and mitral valve replacement.”.

The report generator 100 may extract corresponding finding labels, for example, [Interstitial opacification, uncertain], [consolidation, uncertified], and [Atelectasis] from text about the airways and lungs, such as “Mild pulmonary edema is noted. Left basilar opacification likely reflects atelectasis.”. In this case, the report generator 100 may clearly identify the atelectasis in the text, but it may be difficult to determine whether Interstitial opacity or consolidation exists. In this case, additional information (e.g., uncertain) may be written in the corresponding finding label. The report generator 100 may numerically estimate the uncertainty of the finding label and add the uncertainty score to the corresponding finding label.

The report generator 100 may extract a corresponding finding label, for example, [Pleural effusion] from text about the pleural such as “Small bilateral pleural effusions are present”.

The report generator 100 may extract corresponding finding labels, for example, [Cardiomegaly] and [Aortic atherosclerosis] from text about the Cardiomediastinum and hila such as “The heart is mildly enlarged, CTR 0.61. Calcification of the aortic knob noted.”.

The report generator 100 extracts predefined finding labels, but when findings do not correspond to the finding labels or include unknown findings in the sentences of the radiology report, information included in the corresponding sentence may be written in other items.

Even if the radiology report is automatically generated, the same findings may be expressed in various ways due to the characteristic of the generative language model. For example, radiology reports may express cardiomegaly in various texts as in Table 3. These sentences may be summarized as a single expression, a finding label [Cardiomegaly]. Therefore, the user may quickly identify the main findings detected in the medical image through the finding label set. When evaluating the clinical effectiveness of the system 1, the user quantitatively analyzes metrics such as sensitivity and specificity based on the finding label set, which is output in a consistent form. This allows for evaluation of reliability and reproducibility without the need for further analysis of the high-freedom radiology report.

TABLE 3

| Example of radiology report | Finding label |
|---|---|
| Heart size is enlarged. | Cardiomegaly |
| Cardiac silhouette is widened. | |
| Enlarged cardiomediastinal contour is noted. | |
| Widened cardiac width suggestive of cardiomegaly. | |
| Cardiothoracic ratio is measured up to 0.65, representing | |
| cardiomegaly. | |

Referring back to FIG. 2, the radiology report and finding label set generated by the report generator 100 may be stored in a designated device and may be provided to a user terminal. Here, the initial radiology report generated by the report generator 100 is provided to the user through the user terminal 200, and the final radiology report may be generated through user confirmation. In addition, the initial radiology report generated by the report generator 100 may be edited by a user, and the final radiology report may be generated through user confirmation of the revised report. In particular, the final radiology report on the medical image 10 may be added to the catalog set of the reference storage 120. The final radiology report may be added at the user's request or to the catalog set, or may be selectively added according to the current state of the catalog set (number of cases, feature distribution of cases, and the like).

The report generator 100 may provide an interface for personalizing the format of the radiology report by adjusting the amount of text, a description style, and written content included in the radiology report through setting. Alternatively, report generator 100 may provide an interface for selecting a full version radiology report and a short version radiology report, or may provide various radiology report formats that may be provided, and may generate and provide a radiology report in a format selected by a user.

The radiology report may be provided in, for example, a DICOM basic text SR form. When the user selects a patient's study case for a medical image from the worklist provided through the viewer, the viewer may output a radiology report that is a pre-populated radiology report generated by the system 1. The user may review the radiology report and generate a final radiology report by editing the structure or content of the radiology report through the provided interface.

The finding label set may be provided as a separate radiology report that is distinguished from the radiology report, or may be included in a designated section (e.g., finding label section) of the radiology report. Alternatively, the finding label set 30 may be provided by being included in the DICOM SC generated by the lesion detector 110.

Meanwhile, the report generator 100 may check the analysis result of the non-text format output from the lesion detector 110, the additional information extractor 140, or the measurer 150 and may generate a radiology report including the non-text analysis result by associating the radiology report with the non-text analysis result such as SC image. The report generator 100 may associate text related to various findings such as lesions, organs, and medical devices in the radiology report with the non-text analysis result output from the artificial intelligence analysis model that provides the corresponding findings. The radiology report with the non-text analysis result may be displayed through the viewer of the user terminal 200, and the non-text analysis result corresponding to the text of the radiology report may be provided in an explainable manner. Accordingly, the radiology report with the non-text analysis result may be referred to as an explainable report.

The initial radiology report generated by the report generator 100 may be modified, and the report reviser 160 serves this role. Here, the report reviser 160 is a component introduced to explain a method of revising the initial radiology report and may be included in the report generator 100 to be implemented.

The report reviser 160 may provide an interface through which a user may edit a radiology report generated by the report generator 100. This allows the user to personalize the radiology report.

The report reviser 160 may generate a customized radiology report 20-1 by personalizing the radiology report based on preset user style information. The report reviser 160 may receive a radiology report that may represent a user's preferred radiology report style (text, sentence length, terms used, and the like), and may post-convert the radiology report into a user style radiology report based on the input radiology report. Meanwhile, by adding a radiology report written in the user's preferred style to the catalog set before post-conversion, the report generator 100 may generate a radiology report that automatically reference the user's preferred style at the timing of the generation.

The report reviser 160 may include a language model implemented to generate the radiology report 20-1 by revising the radiology report based on the additional clinical information. The clinical information may include, for example, text data in which a patient's symptoms, test results (e.g., blood test results, function test results, and the like), age, gender, and reason for the test are recorded. The clinical information may include, for example, an additional image other than the target image for which the radiology report is to be generated. The additional image may include a past image taken with an imaging device of the same type as the target image, an image taken with a different type of imaging device, and the like.

The report reviser 160 may add an additional analysis result (e.g., a comparison results with a past image) to the radiology report based on the clinical information, and may revise the sentence of the radiology report using clinical information (e.g., a patient's symptom or test result). The clinical information added to the radiology report or the analysis result added based on the clinical information may be described in a designated section (e.g., a header section) of the radiology report, and reference information may be written in the radiology report to indicate the use of the clinical information. Meanwhile, when the additional clinical information is a past image of a patient, the report reviser 160 may associate the comparison image between the past image and the target image to the radiology report. The comparison image may be provided together with the radiology report through the viewer, and for example, the past image may be provided in the form of a thumbnail to DICOM SC, which is an analysis result of the target image, or may be provided together in a form in which the target image and the past image are divided.

The pre-checker 170 may verify whether the medical image 10 is an image suitable for the artificial intelligence model to analyze before inputting the medical image 10 to the lesion detector 110 or the like. The pre-checker 170 may verify suitability before inputting the medical image 10 to the lesion detector 110, the additional information extractor 140, and the measurer 150, which are artificial intelligence models that perform analysis on the medical image 10. Alternatively, the pre-checker 170 may be implemented in the additional information extractor 140 and implemented as a VQA to check image quality.

The pre-checker 170 may verify whether the quality of the medical image 10 is suitable for analysis by an artificial intelligence model, checking a state such as blur, motion artifact, impact positioning, and the like. The pre-checker 170 may verify whether information on modality acquiring the medical image 10 and/or information included in the metadata of the medical image 10 meet a predetermined criterion. The pre-checker 170 may verify whether the medical image 10 has a distribution similar to the training data used to train the lesion detector 110, the additional information extractor 140, and the measurer 150.

When the pre-checker 170 determines that the medical image is unsuitable, it will prevent the medical image from being input into the artificial intelligence model. The pre-checker 170 may output a notification indicating the medical image's unsuitability for image analysis using an artificial intelligence model. The notification may include a reason for the images' unsuitability, such as blur, motion artifacts, or other quality issues. Also, when it is determined that the medical image is unsuitable for image analysis using an artificial intelligence model, the pre-checker 170 may generate a signal requesting the acquisition of a new medical image for the patient, prompting the system to acquire a new medical image based on the signal.

Alternatively, even if the medical image is determined to be inappropriate by the pre-checker 170, the analysis result is obtained by inputting a medical image into the artificial intelligence model. However, the report generator 100 may generate a radiology report under the assumption that the reliability of the analysis result of the artificial intelligence model on the medical image is low. To this end, the pre-checker 170 may provide the verification result of the medical image 10 to the report generator 100. The report generator 100 may include a note in the medical image explaining the reasons for the image's unsuitability for analysis. The report generator 100 may inform that the reliability of the radiology report based on the unsuitable medical image is low, or may include a disclaimer stating that the quality of the medical image may limit the accuracy of the generated medical image.

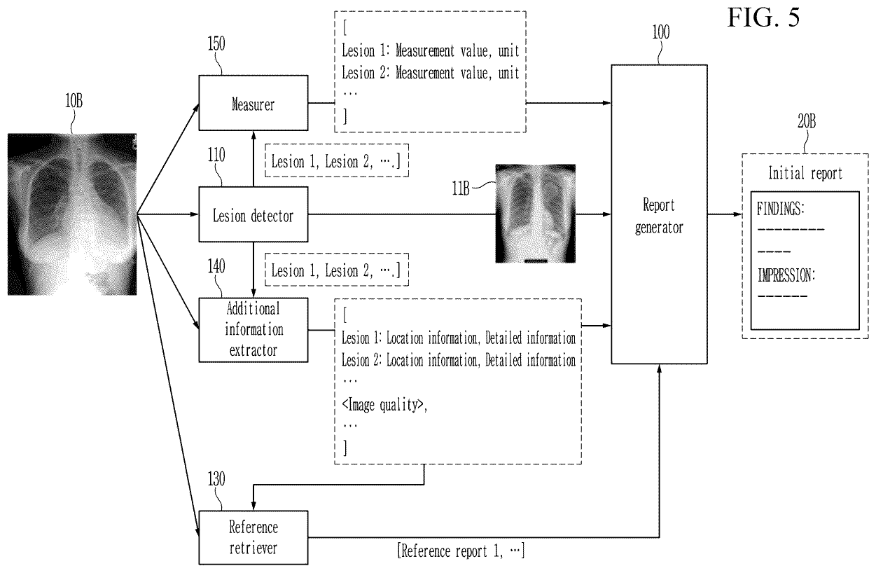

FIG. 5 is a diagram illustrating a radiology report generation method according to an exemplary embodiment.

Referring to FIG. 5, the lesion detector 110, the additional information extractor 140, and the measurer 150 are artificial intelligence analysis models that output analysis results on the medical image 10B, and may be designed to use analysis results of other models in various ways. For example, the lesion detector 110 may play a role of a basic analysis model, and the additional information extractor 140 and the measurer 150 may utilize the analysis result of the lesion detector 110. Alternatively, the lesion detector 110, the additional information extractor 140, and the measurer 150 may be designed as a sequential pipeline to be implemented to sequentially utilize the analysis results of the previous model, or to be implemented to exchange analysis results with other models.

The lesion detector 110 is a lesion list detected in the medical image 10B [lesion 1, lesion 2, . . . ] and an auxiliary image (e.g., SC image) 11B indicating the detected lesion information through a heat map or the like may be output as lesion information. Lesion information, which is a result of analysis by the lesion detector 110, may be provided to help other artificial intelligence analysis models to analyze.

For example, the lesion list [lesion 1, lesion 2, . . . ] detected on medical images 10B may be provided to the additional information extractor 140 and the measurer 150. In this case, an image 11B including lesion information may also be provided to the additional information extractor 140 or the measurer 150.

The additional information extractor 140 may identify a lesion detected in the medical image 10B based on the lesion list, select questions for obtaining additional information on the detected lesion in the question set, or obtain clinical information related to the detected lesion. When a nodule is detected in the medical image 10B, the additional information extractor 140 may select a question such as “Is the nodule is in the upper-right lung zone?” in the question set, and may not select questions about the undetected lesion. Through this, the additional information extractor 140 may obtain additional information on the lesion detected in the medical image 10B through lesion-related questions, that is, location information and/or detailed information, and output additional information including the same.

Alternatively, the additional information extractor 140 may obtain additional information on the medical image 10B by selecting a question as to whether a lesion other than the specific lesion detected by the lesion detector 110 exists in the medical image 10B.

The measurer 150 may identify a lesion detected in the medical image 10B based on the lesion list and measure quantitative information on the lesion. For example, when a nodule is detected in the medical image 10B, the measurer 150 may measure a measurement item (e.g., size) of the nodule that is an interest object in the medical image 10B and generate quantitative information such as <nodule: 3.0, mm>.

Meanwhile, the reference retriever 130 may also receive analysis results provided by at least one of the lesion detector 110, the additional information extractor 140, and the measurer 150 and detect a similar image of the medical image 10B by using the analysis result of the artificial intelligence analysis model. Alternatively, the reference retriever 130 may determine the most similar report from the radiology reports of similar images by using the analysis result of the artificial intelligence analysis model. For example, the reference retriever 130 may compare the similarities between the lesions detected in the medical image 10B and the lesions described in the findings section to determine at least one radiology report having high similarity as the reference report.

The report generator 100 may receive analysis results provided by at least one of the lesion detector 110, the additional information extractor 140, and the measurer 150 and generate a radiology report 20B having a standardized description method and structure for each section. In this case, the report generator 100 may generate a radiology report 20B on the medical image 10B in a structured format based on the analysis result on the medical image 10B with reference to at least one reference report.

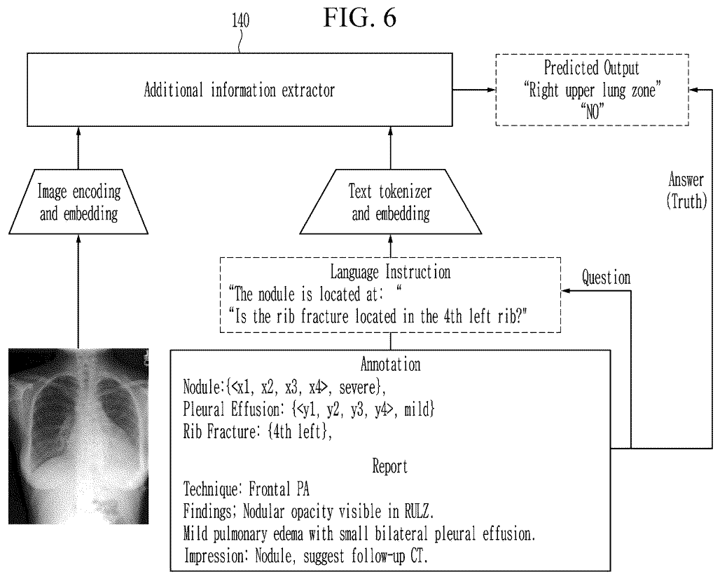

FIG. 6 is a diagram illustrating a visual question and answer (VQA) process of the additional information extractor according to the exemplary embodiment.

Referring to FIG. 6, the additional information extractor 140 may answer to questions for a given medical image through a visual question and answer (VQA) to analyze various additional information such as location information and detailed information on the detected lesion, information on additional findings other than the detected lesion, and quality or metadata of medical images.

For training of the additional information extractor 140, a question and answer pair may be extracted from text data including a radiology report. The medical image may be embedded as an input token of the additional information extractor 140 through image encoding. Questions generated from text data may be embedded as an input token of the additional information extractor 140 through a text tokenizer. The additional information extractor 140 may be trained to generate a correct answer when a medical image and a question are given through visual instruction tuning.

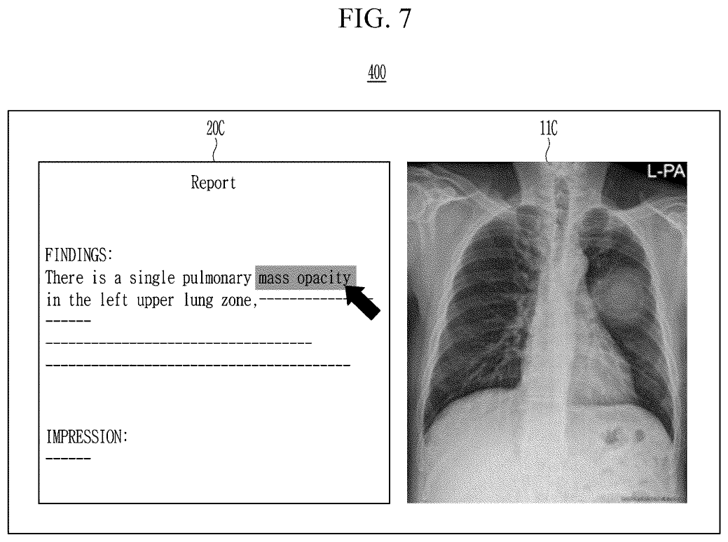

FIG. 7 is a diagram illustrating an example of a radiology report providing method according to an exemplary embodiment.

Referring to FIG. 7, the report generator 100 generates a radiology report based on the analysis result for the medical image, and may provide the radiology report associated with the non-text analysis result.

The user terminal 200 displays a radiology report 20C through the interface 400 provided by the viewer, and may provide a text (e.g., mass opacity), such as a lesion described in the radiology report 20C and an auxiliary image (e.g., SC image) 11C for visually displaying the lesion.

A method of providing the radiology report and the visually displayed analysis result together may be implemented in various ways, and for example, when the user clicks on or hovers over the lesion name in the radiology report 20C, the SC image 11C including lesion information may be displayed together with the radiology report. This allows the user to visually identify the lesion present in the left upper lung zone.

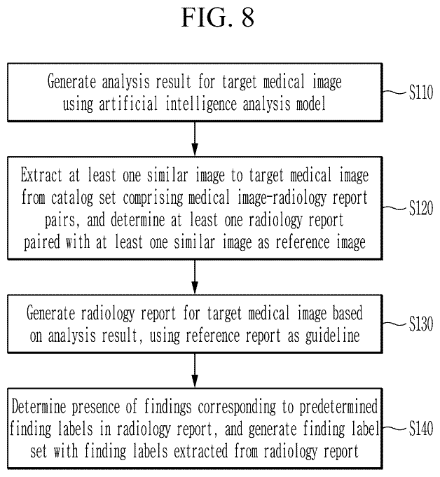

FIG. 8 is a flow diagram of the radiology report generation method according to the exemplary embodiment.

Referring to FIG. 8, the system 1 generates an analysis result for a target medical image using an artificial intelligence analysis model (S110). The analysis result for the target medical image may include predefined lesion information, and the lesion information may include a list of detected lesions and an auxiliary image (e.g., SC image) indicating the information of the detected lesion as a heat map. The analysis result for the target medical image may include additional information extracted from the target medical image, and the additional information may include detailed information on the detected lesion, information on additional findings other than the detected lesion, and information on the quality or metadata of the medical image. The additional information may be obtained through a visual question and answering (VQA) process of extracting answers to questions from a given medical image. Also, the analysis result for the target medical image may include quantitative information (e.g., size, volume, ratio, and number) on an interest object such as a lesion.

The system 1 extracts at least one similar image to the target medical image from the catalog set comprising medical image-radiology report pairs, and determines at least one radiology report paired with the at least one similar image as a reference image (S120).

The system 1 generates a radiology report for the target medical image based on the analysis result, using the reference report as a guideline (S130). The system 1 may generate a radiology report having a standardized description method and structure for each section. The system 1 may associate a non-text analysis result for the target medical image (e.g., an SC image indicating the detected lesion information as a heat map) to the text based-radiology report. Accordingly, when the user clicks on or hovers over a lesion name in the radiology report, the SC image including the lesion information may be displayed together with the radiology report.

The system 1 determines presence of findings corresponding to predetermined finding labels in the radiology report, and generates a finding label set with finding labels extracted from the radiology report (S140). The finding label set indicates whether there are predefined clinical findings in the analysis result of a medical image and may be used to evaluate the clinical validity of the radiology report. The finding label set may be provided as a separate radiology report distinct from the radiology report, or may be included in a designated section (e.g., finding label section) of the radiology report.

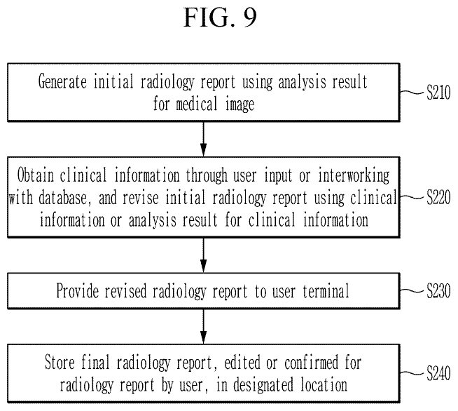

FIG. 9 is a flowchart of a method of revising a radiology report according to the exemplary embodiment.

Referring to FIG. 9, the system 1 generates an initial radiology report using the analysis result for the medical image (S210).

The system 1 obtains clinical information through user input or interworking with a database, and revises the initial radiology report using the clinical information or the analysis result for the clinical information (S220). The clinical information may include, for example, text data detailing a patient's symptoms, test results (e.g., blood test, function test results), age, gender, and reason for the test. The clinical information may include, for example, an additional image other than the target image for which the radiology report is being generated. The additional image may include a past image taken with the same type of imaging device as the target image, or an image taken with a different type of imaging device from the target image. The system 1 may add an additional analysis result (e.g., comparison result with a past image) to the radiology report based on the clinical information, or may revise the sentence of the radiology report using clinical information (e.g., a patient's symptom or test result). The database of the medical institution may include a medical image storage and transmission system (PACS), an electronic medical record (EMR), an electronic health record (EHR), and the like.

The system 1 provides the revised radiology report to the user terminal 200 (S230). The system 1 stores the final radiology report, edited or confirmed for the radiology report by a user, in a designated location (S240). The final radiology report may be added to the catalog set with medical image-radiology report pairs according to user selection or determination of the system 1.

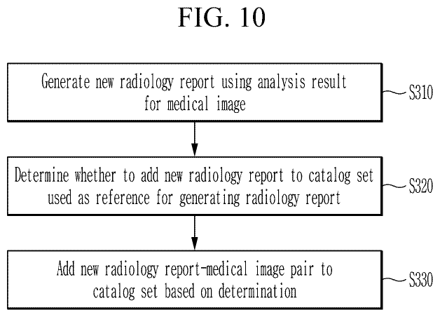

FIG. 10 is a flowchart of a method of managing a catalog set according to the exemplary embodiment.

Referring to FIG. 10, the system 1 generates a new radiology report using the analysis result for the medical image (S310).

The system 1 determines whether to add the new radiology report to the catalog set used as a reference for generating the radiology report (S320). According to the exemplary embodiment, the system 1 may determine whether it is useful to add a new radiology report to the catalog set. The system 1 may determine the usefulness of the new radiology report by measuring an information gain obtained by adding the new radiology report to the catalog set. According to another exemplary embodiment, the system 1 may embed radiology reports as feature representation vectors and evaluate the similarity between the radiology reports stored in the catalog set and the new radiology report. Further, the system 1 may add a new radiology report determined to have low similarity with reference reports stored in the catalog set to the catalog set based on the similarity distance between the new radiology report and the reference report. According to another exemplary embodiment, the system 1 may determine whether to add the radiology report generated by report generator 100 to the catalog set based on the characteristics of the catalog set such as the number of cases included in the catalog set and the feature distribution of the cases (e.g., lesion, and other findings distribution) included in the catalog set.

The system 1 adds a new radiology report-medical image pair to the catalog set based on the determination (S330). When the catalog set is composed of M cases, the system 1 may discard one case among the M cases included in the catalog set and add a new radiology report. The system 1 may discard a case similar to the other case among the M cases included in the catalog set. The system 1 may discard a case having the lowest information gain among the M cases included in the catalog set.

The system 1 of the present disclosure may include one or more processors, a memory loading a computer program executed by the processor, a storage device storing computer programs and various data, and a communication interface, and various components may be further included. The processor may be a processor of various forms that process instructions included in a computer program, and may include, for example, at least one of a Central Processing Unit (CPU), a Micro Processor Unit (MPU), a Micro Controller Unit (MCU), a Graphic Processing Unit (GPU), or any type of processor well known in the technical field of the present disclosure. The memory stores various types of data, instructions, and/or information. The memory may load the corresponding computer program from the storage device such that instructions described to execute the operation of the present disclosure are processed by the processor. The memory may be, for example, a read only memory (ROM), a random access memory (RAM), and the like

The storage device may non-temporarily store computer programs and various types of data. The storage device may include a nonvolatile memory, such as a Read Only Memory (ROM), an Erasable Programmable ROM (EPROM), an Electrically Erasable Programmable ROM (EEPROM), a flash memory or the like, a hard disk, a removable disk, or any type of computer-readable recording medium well known in the art to which the present disclosure pertains. The communication interface may be a wired/wireless communication module supporting wired/wireless communication. The computer program includes instructions executed by the processor, is stored in a non-transitory computer readable storage medium, and instructions cause the processor to execute the operation of the present disclosure.

The exemplary embodiments of the present disclosure described above are not only implemented through the apparatus and method, but may also be implemented through programs that realize functions corresponding to the configurations of the exemplary embodiment of the present disclosure, or through recording media on which the programs are recorded.

Although an exemplary embodiment of the present disclosure has been described in detail, the scope of the present disclosure is not limited by the exemplary embodiment. Various changes and modifications using the basic concept of the present disclosure defined in the accompanying claims by those skilled in the art shall be construed to belong to the scope of the present disclosure.

What is claimed is:

1. A system for generating a radiology report, the system comprising:
a memory; and
a processor for executing instructions stored in the memory,
wherein the processor is configured to:
obtain an analysis result for a target medical image using an artificial intelligence analysis model;
extract at least one similar image to the target medical image from a catalog set comprising medical image-radiology report pairs;
determine at least one radiology report paired with the at least one similar image as a reference report; and
generate a radiology report for the target medical image based on the analysis result, using the reference report as a guideline.

2. The system of claim 1, wherein the processor is configured to:
determine presence of findings corresponding to predetermined finding labels in the radiology report; and
generate a finding label set with finding labels extracted from the radiology report, and
wherein the finding label set is provided as a separate report distinct from the radiology report, or included in a designated section of the radiology report.

3. The system of claim 1, wherein the processor is configured to:
obtain clinical information through user input or interworking with a database of a medical institution; and
revise the radiology report using the clinical information or an analysis result of the clinical information.

4. The system of claim 1, wherein the processor is configured to store a final radiology report, edited or confirmed for the radiology report by a user, in a designated location.

5. The system of claim 1, wherein the processor is configured to:
determine whether to add the radiology report to the catalog set; and
add a pair of the radiology report and the target medical image to the catalog set based on the determination.

6. The system of claim 1, wherein the analysis result comprises lesion information detected in the target medical image.

7. The system of claim 6, wherein the analysis result further comprises additional information extracted from the target medical image, and
the additional information comprises at least one of detailed information on the detected lesion, information on additional findings other than the detected lesion, quality information on the target medical image, or information on metadata for the target medical image.

8. The system of claim 7, wherein the processor is configured to generate the additional information through visual question answering process, which extracts answers to questions in the target medical image.

9. The system of claim 8, wherein the processor is configured to:
select a question set related to the target medical image or an analysis result of the target medical image from a question bank having questions; and
extract an answer to each question included in the question set to generate the additional information.

10. The system of claim 6, wherein the analysis result further comprises quantitative information on an interest object present in the target medical image.

11. The system of claim 1, wherein the processor is configured to associate a non-text analysis result for the target medical image with the radiology report.

12. A method of a radiology report generation by a system, the method comprising:
obtaining an analysis result for a target medical image using an artificial intelligence analysis model;
extracting at least one similar image to the target medical image from a catalog set comprising medical image-radiology report pairs;
determining at least one radiology report paired with the at least one similar image as a reference report; and
generating a radiology report for the target medical image based on the analysis result, using the reference report as a guideline.

13. The method of claim 12, further comprising:
determining presence of findings corresponding to predetermined finding labels in the radiology report; and
generating a finding label set with finding labels extracted from the radiology report.

14. The method of claim 12, further comprising:
obtaining clinical information through user input or interworking with a database of a medical institution; and
revising the radiology report using the clinical information or an analysis result of the clinical information.

15. The method of claim 12, further comprising:
storing a final radiology report, edited or confirmed for the radiology report by a user, in a designated location.

16. The method of claim 12, further comprising:
determining whether to add the radiology report to the catalog set; and
adding a pair of the radiology report and the target medical image to the catalog set based on the determination.

17. The method of claim 12, wherein the analysis result comprises at least one of lesion information detected in the target medical image, additional information extracted from the target image, or quantitative information on an interest object present in the target medical image, and
the additional information comprises at least one of detailed information on the detected lesion, information on additional findings other than the detected lesion, quality information on the target medical image, or information on metadata for the target medical image.

18. The method of claim 17, wherein the obtaining the analysis result comprises:
selecting a question set related to the target medical image or an analysis result of the target medical image from a question bank having questions, and extracting an answer to each question included in the question set to generate the additional information.

19. The method of claim 12, further comprising:
associating a non-text analysis result for the target medical image with the radiology report.

20. A computer program stored in a computer-readable recording medium, the computer program comprising instructions to cause a processor configured to:
obtain an analysis result for a target medical image using an artificial intelligence analysis model;
extract at least one similar image to the target medical image from a catalog set comprising medical image-radiology report pairs;
determine at least one radiology report paired with the at least one similar image as a reference report; and
generate a radiology report for the target medical image based on the analysis result, using the reference report as a guideline.
